(19) United States
(12) Patent Application Publication
(10) Pub. No.: US 2023/0310556 A1
(43) Pub. Date: Oct. 5, 2023

(54) PREVENTION AND TREATMENT OF DISEASES USING PHYTASE

(71) Applicant: ACCESS NUTRIENTS INC., Calgary (CA)

(72) Inventor: Amy Frances PUZEY, Invermere (CA)

(21) Appl. No.: 18/025,014

(22) PCT Filed: Dec. 21, 2022
(86) PCT No.: PCT/CA2022/051877

§ 371 Date: Mar. 7, 2023

Related U.S. Application Data
(60) Provisional application No. 63/293,234, filed on Dec. 23, 2021.

(51) Int. Cl. A61K 38/46; A61P 1/00; A61K 33/30; A61K 38/28; A61P 3/10; A61P 37/00; A61P 5/14; A61P 35/00; A61P 7/06; A61P 19/02; A61P 29/00

(52) U.S. Cl. CPC A61K 38/465; A61P 1/00; C12Y 301/03008; A61K 33/30; A61K 38/28; A61P 3/10; A61P 37/00; A61P 5/14; A61P 35/00; A61P 7/06; A61P 19/02; A61P 29/00

(57) ABSTRACT

The present disclosure provides methods for preventing or treating an immune disease, an autoimmune disease, and/or a disease caused by viral infection in a subject. The method comprises administering phytase to the subject, optionally with zinc.

### FIELD

The present disclosure relates generally to methods of preventing and/or treating diseases, particularly diseases caused by viral infections, and immune and autoimmune diseases, using phytase.

### BACKGROUND

The following paragraphs are not an admission that anything discussed in them is prior art or part of the knowledge of persons skilled in the art.

An immune system disorder is caused by abnormally low activity or over activity of the immune system. An autoimmune disease is caused by the immune system attacking and damaging its own tissues. There are over 100 known autoimmune diseases, the more common ones being lupus, rheumatoid arthritis, Type 1 diabetes, Crohn's disease, and ulcerative colitis and other inflammatory diseases. Some research has suggested that Type 2 diabetes may be an autoimmune disease. Other research has suggested that Long COVID may be an autoimmune disease.

A viral disease is a condition caused by a virus entering a host cell and using the host cells to replicate the virus' RNA and DNA to reproduce. There are several types of viral diseases depending on the underlying virus, such as COVID-19, Long COVID, influenza, the common cold, mumps, chickenpox, and herpes.

Phytate or phytic acid is the principal storage form of phosphorus in many plant tissues, especially bran and seeds. Phytic acid also referred to as inositol hexakisphosphate (IP6) or inositol polyphosphate. Phytase is a phosphatase enzyme that catalyzes the hydrolysis of phytic acid.

### INTRODUCTION

The following introduction is intended to introduce the reader to this specification but not to define any invention. One or more inventions may reside in a combination or sub-combination of the instrument elements or method steps described below or in other parts of this document. The inventors do not waive or disclaim their rights to any invention or inventions disclosed in this specification merely by not describing such other invention or inventions in the claims.

The present disclosure provides methods for preventing or treating immune diseases, autoimmune diseases, and/or diseases caused by viral infections using phytase, optionally with zinc. The inventors have discovered that an appropriate amount of phytase, with or without an appropriate amount of zinc, can decease the susceptibility of contracting, decreasing the occurrence, and/or decreasing the effects of an immune disease, an autoimmune disease, and/or a disease caused by viral infection. Without being bound by theory, the inventors believe that an appropriate amount of phytase administered to a subject, optionally with an appropriate amount of zinc: 1) inhibits, disables, or reduces the amount of cytokine IL-18, which is involved in autoimmune diseases, particularly autoimmune inflammation diseases such as Crohn's disease, Irritable Bowel Syndrome (IBS), ulcerative colitis, Type 1 diabetes, Type 2 diabetes, COVID-19, Long COVID, and other types of inflammatory diseases; 2) inhibits, disables, or reduces the amount of cytokine IL-6, which is involved in autoimmune diseases such as Crohn's disease, Irritable Bowel Syndrome (IBS), rheumatoid arthritis, prostate cancer, systemic lupus erythematosus, Alzheimer's disease, depression, atherosclerosis, Multiple Sclerosis, ulcerative colitis, Type 1 diabetes, Type 2 diabetes, COVID-19, Long COVID, and other types of inflammatory diseases; 3) inhibits, disables, or reduces the amount of cytokine TNF-α, which is involved in autoimmune diseases, particularly autoimmune inflammation diseases such as Crohn's disease, Irritable Bowel Syndrome (IBS), ulcerative colitis, psoriasis, ankylosing spondylitis, rheumatoid arthritis, Type 1 diabetes, Type 2 diabetes, COVID-19, Long COVID, and other types of inflammatory diseases; and/or 4) inhibits viral replication of a viral infection, including in particular, the SARS-CoV2 or COVID-19 virus and all of its variations through increased zinc absorption and subsequent zinc homeostasis. Importantly, the inventors discovered that an amount less than or greater than an appropriate amount of phytase administered to a subject, optionally with zinc, does not sufficiently prevent or treat an immune disease, autoimmune disease, and/or disease caused by a viral infection.

The present disclosure also discusses compositions comprising phytase, optionally with zinc, and uses of phytase optionally with zinc, for preventing or treating an immune disease, an autoimmune disease, and/or a disease caused by viral infection.

The present disclosure provides a method for preventing or treating an insulin-related disorder in a subject, comprising administering phytase to the subject. Optionally, the phytase is administered with insulin.

The present disclosure also provides a method for preventing or treating Long COVID in a subject, comprising administering phytase to the subject. Optionally, the phytase is administered with zinc.

The present disclosure also provides a method for preventing or treating Crohn's disease in a subject, comprising administering phytase to the subject.

The present disclosure also provides a method for preventing or treating Ulcerative Colitis in a subject, comprising administering phytase to the subject.

Other aspects and features of the present disclosure will become apparent to those ordinarily skilled in the art upon review of the following description of specific examples in conjunction with the accompanying figures.

### BRIEF DESCRIPTION OF THE DRAWINGS

Examples of the presently disclosed methods will now be described, by way of example only, with reference to the attached Figures.

FIG. 1 is a graph showing the effect of the herein disclosed phytase administration on pre-treatment on bodyweight.

FIG. 2 is a graph showing the effect of the herein disclosed phytase administration on bodyweight shown as a percentage change.

FIG. 3 panels A to D are graphs showing the effect of the herein disclosed phytase administration pre-treatment on the peritoneal IL-1β cytokine (FIG. 3A); IL-6 cytokine (FIG. 3B); IL-10 cytokine (FIG. 3C); and TNF-α cytokine (FIG. 3D) response to LPS challenge.

FIG. 4 is a graph showing the effect of the herein disclosed phytase administration on IL-1β.

FIG. 5 is a graph showing the effect of the herein disclosed phytase administration on IL-6.

FIG. 6 is a graph showing the effect of the herein disclosed phytase administration on TNF-α.

### DETAILED DESCRIPTION

Generally, the present disclosure provides a method for preventing or treating an immune disease, an autoimmune disease, and/or a disease caused by viral infection in a subject, comprising: administering from about 400 FTU to about 5000 FTU of phytase per day to the subject.

The present disclosure also provides a method for preventing or treating an insulin-related disorder in a subject, comprising: administering from about 400 FTU to about 5000 FTU of phytase per day to the subject.

The present disclosure also provides a method for preventing or treating Long COVID in a subject, comprising: administering from about 400 FTU to about 5000 FTU of phytase per day to the subject. Optionally, further comprising administering from about 10 mg to about 50 mg of zinc to the subject per day.

The present disclosure also provides a method for preventing or treating Crohn's disease in a subject, comprising: administering from about 400 FTU to about 5000 FTU of phytase per day to the subject.

The present disclosure also provides a method for preventing or treating Ulcerative Colitis in a subject, comprising: administering from about 400 FTU to about 5000 FTU of phytase per day to the subject.

In the context of the present disclosure, an immune disease is characterized by abnormally low activity or over activity of the immune system. Instances in which the immune system is over activity, the body attacks and damages its own tissue, which results in autoimmune diseases. Immune diseases may decrease the body's ability to fight invaders, causing vulnerability to infections. Examples of an immune disease include diseases associate with elevated levels of IL-1β cytokine, IL-6 cytokine, TNF-α cytokine, and/or primary and secondary acquired immunodeficiency, i.e. HIV,. Examples of an autoimmune disease include Crohn's disease, Irritable Bowel Syndrome (IBS), rheumatoid arthritis, prostate cancer, systemic lupus erythematosus, Alzheimer's disease, depression, atherosclerosis, Multiple Sclerosis, ulcerative colitis, Type 1 diabetes, Type 2 diabetes, COVID-19, Long COVID, and other types of inflammatory diseases. An inflammatory disease refers to a disorder resulting when the immune system causes inflammation by mistakenly attacking its body's own cells or tissues. Examples of an inflammation disease include Crohn's disease, Irritable Bowel Syndrome (IBS), rheumatoid arthritis, Type 1 diabetes, Type 2 diabetes, lupus erythematosus, fatty liver disease, Alzheimer's disease, and Parkinson's disease. In some examples according to the present disclosure, the immune disease is associated with elevated levels of IL-1β cytokine relative to normal levels of IL-1β cytokine in a subject that does not have the immune disease. In some examples according to the present disclosure, the immune disease is associated with elevated levels of IL-6 cytokine relative to normal levels of IL-6 cytokine in a subject that does not have the immune disease.

In some examples according to the present disclosure, the immune disease is associated with elevated levels of TNF-α cytokine relative to normal levels of TNF-α cytokine in a subject that does not have the immune disease.

In the context of the present disclosure, a disease caused by viral infection refers to any illness or health condition caused by a virus. Antiviral activity may be by prevention of viral entry into the cell through upregulation of zinc antiviral proteins and/or through inhibition of viral replication. Examples of a disease caused by viral infection include COVID-19, Long COVID, influenza, the common cold, mumps, chickenpox, herpes, Epstein Barr, and Merkel cell virus.

In the context of the present disclosure, an insulin-related disorder refers to any physiological condition where the natural hormone insulin becomes less effective at lowering blood sugars characterized by, for example, high blood sugar, insulin resistance, and/or relative lack of insulin. The resulting increase in blood glucose may raise levels outside the normal range and cause adverse health effects such Type I diabetes and Type 2 diabetes. Symptoms of Type I diabetes include frequent urination, increased thirst, increased hunger, weight loss, and other serious complications. Other symptoms of Type I diabetes include blurry vision, tiredness, and slow wound healing. Symptoms of Type II diabetes include increased thirst, frequent urination, and unexplained weight loss. Other symptoms of Type II diabetes include increased hunger, feeling tired, and sores that do not heal.

In the context of the present disclosure, Long COVID refers to any condition characterized by long-term health problems persisting or appearing after the typical recovery period of COVID-19. Common symptoms of Long COVID are fatigue and memory problems. Other symptoms such as malaise, headaches, shortness of breath, anosmia, parosmia, muscle weakness, low-grade fever, and cognitive dysfunction, have also been associated with Long COVID.

In the context of the present disclosure, Crohn's disease refers to a type of inflammatory bowel disease (IBD) that may affect any segment of the gastrointestinal tract. Crohn's disease includes ileocolitis, ileitis, Gastroduodenal Crohn's Disease, Jejunoileitis, and/or Crohn's (Granulomatous) Colitis. Symptoms of Crohn's disease include abdominal pain, diarrhea, fever, abdominal distension, and weight loss.

In the context of the present disclosure, ulcerative colitis refers to a condition that results in inflammation and ulcers of the colon and rectum. Ulcerative Colitis includes Ulcerative proctitis, Proctosigmoiditis, Left-sided colitis, and/or Pancolitis. Symptoms of ulcerative colitis include abdominal pain and diarrhea mixed with blood (hematochezia). Other symptoms of ulcerative colitis include weight loss, fever, and anemia.

Preventing or treating a disease or disorder refers to the action that can: 1) keep the disease from occurring or one or more symptoms of the disease from occurring; 2) reverse or relieve the disease or one or more symptoms of the disease; 3) inhibit the progress, development and/or spread of the disease or the progression and/or spread of one or more symptoms of the disease; 4) palliate one or more symptoms of the disease; and/or 5) prevent the reoccurrence of the disease or one or more symptoms of the disease.

In the context of the present disclosure, phytase refers to any phosphatase enzyme that catalyzes the hydrolysis of phytic acid. Phytase may be sourced as phytase expressed from Aspergillus Niger, Aspergillus Ficuum, or the new protein tyrosine phosphatase-like phytase expressed from the gut bacteria of ruminant animals. Sourcing phytase from Aspergillus Ficuum may be advantageous because it has a broad pH range, which is helpful for digestion in the human gastrointestinal (GI) system. The phytase may be formulated for oral administration, for example, in dry or liquid form within a capsule, or intravenously. In the context of the present disclosure, zinc refers to any solid or liquid form of zinc that is formulated for administration to a subject.

The total amount of the herein disclosed phytase administered to a subject per day may be any appropriate amount to sufficiently prevent or treat an immune disease, an autoimmune disease, and/or a disease caused by viral infection in a subject, for example, from about 400 FTU to about 5000 FTU; about 400 FTU; about 500 FTU; about 600 FTU; about 700 FTU; about 800 FTU; about 900 FTU; about 1000 FTU; about 1500 FTU; about 2000 FTU; about 2500 FTU; about 3000 FTU; about 3500 FTU; about 4000 FTU; about 4500 FTU; about 5000; or from any one of the recited FTUs to any other of the recited FTUs. One phytase unit (FTU) is the activity of phytase required to liberate 1 μmol of inorganic phosphorus per minute at pH 5.5 from an excess of 15 M sodium phytate at 37° C. The inventors discovered that administering an amount of phytase to a subject per day less than or greater than an appropriate amount, for example, less than 400 FTU or greater than 5000 FTU, did not sufficiently prevent or treat an immune disease, autoimmune disease and/or disease caused by a viral infection.

An appropriate amount of the herein disclosed phytase administered to a subject per day may be an amount that causes a subject's IL-1β cytokine level, IL-6 cytokine level, and/or TNF-α cytokine level to decrease. In some examples according to the present disclosure, an appropriate amount of phytase administered to a subject per day may cause a subject's IL-1β cytokine level to decrease, for example, by from about 10% to about 70%; about 10%; about 20%; about 30%; about 40%; about 50%; about 60%; about 70%; or from any one of the recited percentages to any other of the recited percentages, following from about 72 hours to about 90 days of administration. In some examples according to the present disclosure, an appropriate amount of phytase administered to a subject per day may cause a subject's IL-6 cytokine level to decrease, for example, by from about 1% to about 10%; about 1%; about 2%; about 3%; about 4%; about 5%; about 6%; about 7%; about 8%; about 9%; about 10%; or from any one of the recited percentages to any other of the recited percentages, following from about 72 hours to about 90 days of administration. In some examples according to the present disclosure, an appropriate amount of phytase administered to a subject per day may cause a subject's TNF-α cytokine level to decrease, for example, by from about 1% to about 40%; about 1%; about 2%; about 3%; about 4%; about 5%; about 6%; about 7%; about 8%; about 9%; about 10%; about 15%; about 20% about 25%; about 30%; about 35%; about 40%; or from any one of the recited percentages to any other of the recited percentages, following from about 72 hours to about 90 days of administration.

The amount of the herein disclosed phytase administered to a subject in a single dose may be from about 190 FTY to about 5000 FTU, for example, about 190 FTU; about 200 FTU; about 300 FTU; about 400 FTU; about 500 FTU; about 600 FTU; about 700 FTU; about 800 FTU; about 900 FTU; about 1000 FTU; about 1500 FTU; about 2000 FTU; about 2500 FTU; about 3000 FTU; about 3500 FTU; about 4000 FTU; about 4500 FTU; about 5000 FTU; or from any one of the recited FTUs to any other of the recited FTUs. The amount of phytase formulated in a single dose may be about 800 FTU, for example when decreasing the number of phytase administrations per day is desired.

The herein disclosed phytase may be administered to a subject from about once per day to about 26 times per day or more, for example, once per day; twice per day; three times per day; four times for day; five times per day; six times per day; seven times per day; eight times per day; nine times per day; 10 times per day; 11 times per day; 12 times per day; 13 times per day; 14 times per day; 15 times per day; 16 times per day; 17 times per day; 18 times per day; 19 times per day; 20 times per day; 21 times per day; 22 times per day; 23 times per day; 24 times per day; 25 times per day; 26 times per day; or more. In some examples according to the present disclosure, phytase is administered twice per day, for example, one dose in the morning of the day and a second dose in the afternoon of the day, when convenience of administration is desired. In some examples according to the present disclosure relating to preventing to treating insulin-related disorders, phytase is administered when the blood sugar level of a subject is higher than normal, for example, within 60 minutes of determining the blood sugar level.

The herein disclosed phytase may be administered to a subject for a sufficient length of time to prevent or treat an immune disease, an autoimmune disease, and/or a disease caused by viral infection, for example, for a duration of from about 1 day to about 360 days,; for the lifetime of the subject; about 1 day; about 5 days; about 7 days; about 10 days; about 14 days; about 21 days; about 28 days; about 30 days; about 60 days; about 90 days; about 120 days; about 180 days; about 210 days; about 240 days; about 270 days; about 300 days; about 360 days; about 365 days; or from any one of the recited days to any other of the recited days.

The herein disclosed phytase may be administered to a subject in proximity to the beginning of a meal, for example, from about 60 minutes prior to beginning a meal to about 180 minutes after beginning a meal. Proximity to a meal refers to a period near in time to the beginning of a meal. In the context of the present disclosure, meal refers to traditional meals and meal times; however, these also include the ingestion of any substance regardless of size and/or timing. Alternatively, phytase may be administered in remoteness to the beginning of a meal, for example, greater than 60 minutes prior to beginning a meal and greater than 180 minutes after beginning a meal. In some examples according to the present disclosure relating to preventing or treating insulin-related disorders, phytase is administered in proximity to beginning a meal, for example when monitoring and adjusting the amount of phytase in relation to the sugar content of the meal is desired.

The herein disclosed phytase may be administered in solid form, for example powder, or in liquid form. The solid or liquid form may be formulated within a suitable capsule. Phytase may be administered orally or intravenously. Phytase may be administered as part of a composition. The composition may further comprise a pharmaceutically acceptable carrier or excipient, zinc, insulin, micronutrients, vitamins, and/or minerals.

The herein disclosed amount, dose, and/or duration of the administration of phytase to a subject may be adjusted to cause the amount of medicine and/or treatment administered to the subject alone, e.g., without phytase and with or without zinc, to prevent or treat an immune disease, an autoimmune disease, and/or a disease caused by viral infection to be reduced by from about 10% to about 95%, for example, about 10%; about 15%; about 20%; about 25%; about 30%; about 35%; about 40%; about 45%; about 50%; about 55%; about 60%; about 65%; about 70%; about 75%; about 80%; about 85%; about 90%; about 95%; 100%; or from any one of the recited percentages to any other of the recited percentages. The reduction may be observed following from about 1 day to about 180 days of the herein disclosed administration, for example, about 1 day; about 2 days; about 3 days; about 4 days; about 5 days; about 6 days; about 7 days; about 8 days; about 9 days; about 10 days; about 11 days; about 12 days; about 13 days; about 14 days; about 21 days; about 28 days; about 30 days; about 35 days; about 40 days; about 45 days; about 50 days; about 60 days; about 120 days; about 180 days; or from any one of the recited days to any other of the recited days. For inflammation related disorders, the medicine and/or treatment may be insulin, Remicade™, a biologics treatment such as Humira™, a steroid treatment such as Prednisone, Cimzia, Enbrel, and/or Simponi/Simponi Aria.

The herein disclosed amount, dose, and/or duration of the administration of phytase to a subject may be adjusted to cause the energy level of the subject to increase compared to the subject's energy level without phytase administration, with or without zinc, by from about 10% to 100%, for example, about 10%; about 15%; about 20%; about 25%; about 30%; about 35%; about 40%; about 45%; about 50%; about 55%; about 60%; about 65%; about 70%; about 75%; about 80%; about 85%; about 90%; about 95%; 100% or from any one of the recited percentages to any other of the recited percentages. The increase of energy may be observed following from about 1 day to about 180 days of the herein disclosed administration, for example, about 1 day; about 2 days; about 3 days; about 4 days; about 5 days; about 6 days; about 7 days; about 8 days; about 9 days; about 10 days; about 11 days; about 12 days; about 13 days; about 14 days; about 21 days; about 28 days; about 30 days; about 35 days; about 40 days; about 45 days; about 50 days; about 60 days; about 120 days; about 180 days; or from any one of the recited days to any other of the recited days.

The herein disclosed amount, dose, and/or duration of the administration of phytase to a subject may be adjusted to cause the pain level of the subject to decrease compared to the subject's pain level without phytase administration, with or without zinc, by from about 10% to 100%, for example, about 10%; about 15%; about 20%; about 25%; about 30%; about 35%; about 40%; about 45%; about 50%; about 55%; about 60%; about 65%; about 70%; about 75%; about 80%; about 85%; about 90%; about 95%; 100% or from any one of the recited percentages to any other of the recited percentages. The decrease in pain may be observed following from about 1 day to about 180 days of the herein disclosed administration, for example, about 1 day; about 2 days; about 3 days; about 4 days; about 5 days; about 6 days; about 7 days; about 8 days; about 9 days; about 10 days; about 11 days; about 12 days; about 13 days; about 14 days; about 21 days; about 28 days; about 30 days; about 35 days; about 40 days; about 45 days; about 50 days; about 60 days; about 120 days; about 180 days; or from any one of the recited days to any other of the recited days.

Optionally, zinc may be administered to a subject with phytase in an appropriate amount to, with phytase, sufficiently prevent or treat an immune disease, an autoimmune disease, and/or a disease caused by viral infection in a subject, for example, from about 10 mg to about 50 mg per day; about 10 mg per day; about 15 mg per day; about 20 mg per day; about 25 mg per day; about 30 mg per day; about 35 mg per day; about 40 mg per day; about 45 mg per day; about 50 mg per day; or from any one of the recited amounts to any other of the recited amounts. Zinc may be administered to the subject with phytase when, for example, preventing or treating a disease caused by viral infection is desirable. Without being bound by theory, the inventors believe that, while zinc inhibits viral replication, it is difficult to achieve zinc homeostasis without completely disabling, or dephosphorylating IP6 (phytate). Disabling IP6 (phytate), or dephosphorylating it could disable the autoimmune reaction and a cytokine storm, as well as all necroptosis (cell death) that ensues after viral infection. In the context of the present disclosure, a cytokine storm, also called hypercytokinemia, refers to when a physiological reaction in humans and other animals in which the innate immune system causes an uncontrolled and excessive release of pro-inflammatory signaling cytokines. Administering an appropriate amount of phytase to disable most or all IP6 improves zinc status and zinc homeostasis. Zinc homeostasis is required to prevent viral infection and viral replication of COVID-19 as well as other viruses. The Applicant postulates that, in view of the factors mentioned above, disease caused by viral infection, as well as the cytokine storm and organ damage induced by a SARS-CoV2 viral infection may be prevented and/or treated by an appropriate combination of phytase and zinc.

The amount of the herein disclosed zinc administered to a subject with phytase in a single dose may be from about 10 mg to about 50 mg, for example, about 10 mg; about 15 mg; about 20 mg; about 25 mg; about 30 mg; about 35 mg; about 40 mg; about 45 mg; about 50 mg; or from any one of the recited amounts to any other of the recited amounts. In some examples according to the present disclosure, the amount of zinc in a single dose is selected such that the total amount of zinc administered to the subject per day is split evenly between a number of doses that corresponds or equates with the number of doses of phytase administered to the subject in the day.

The herein disclosed zinc may be administered to a subject with phytase from about once per day to about 26 times per day or more, for example, once per day; twice per day; three times per day; four times for day; five times per day; six times per day; seven times per day; eight times per day; nine times per day; 10 times per day; 11 times per day; 12 times per day; 13 times per day; 14 times per day; 15 times per day; 16 times per day; 17 times per day; 18 times per day; 19 times per day; 20 times per day; 21 times per day; 22 times per day; 23 times per day; 24 times per day; 25 times per day; 26 times per day; or more. In some examples according to the present disclosure, the number of doses of zinc administered per day is selected to correspond or equate with the herein disclosed number of doses of phytase administered to the subject in a day.

The herein disclosed zinc may be administered to a subject for a sufficient length of time to, with phytase, prevent or treat an immune disease, an autoimmune disease, and/or a disease caused by viral infection, for example, for a duration of from about 1 day to about 360 days; for the lifetime of the subject; about 1 day; about 5 days; about 7 days; about 10 days; about 14 days; about 21 days; about 28 days; about 30 days; about 60 days; about 90 days; about 120 days; about 180 days; about 210 days; about 240 days; about 270 days; about 300 days; about 360 days; about 365 days; or from any one of the recited days to any other of the recited days.

The herein disclosed zinc may be administered in combination with the phytase, concurrently with the phytase, or non-concurrently with the phytase. Concurrently refers to administration at the same time but not necessarily at the same part of the body. In some examples according to the present disclosure, zinc is administered from about 20 minutes prior to the administration of phytase to about 120 minutes after the administration of phytase. Alternatively, zinc is administered greater than 20 minutes prior to the administration of phytase to about greater than 120 minutes after the administration of phytase. The administration of phytase and zinc may be non-concurrent when, for example, the phytase dose(s) and/or zinc dose(s) require adjustment without similarly adjusting the other of the phytase dose(s) or zinc dose(s), is desirable. Without being bound by theory, the inventors believe that, as phytate affects the endogenous pool of zinc, to achieve zinc homeostasis, most or all of the phytate (IP6) should be degraded in the gut so as to achieve zinc homeostasis, which would require adjusting the phytase and/or zinc dose(s).

The herein disclosed amount, dose, and/or duration of the administration of zinc to a subject may be adjusted to, with phytase, cause the amount of medicine and/or treatment administered to the subject alone, e.g., without phytase and zinc, to prevent or treat an immune disease, an autoimmune disease, and/or a disease caused by viral infection to be reduced from about 10% to about 95%, for example, about 10%; about 15%; about 20%; about 25%; about 30%; about 35%; about 40%; about 45%; about 50%; about 55%; about 60%; about 65%; about 70%; about 75%; about 80%; about 85%; about 90%; about 95%; 100%; or from any one of the recited percentages to any other of the recited percentages. The reduction may be observed following from about 1 day to about 180 days of the herein disclosed administration, for example, about 1 day; about 2 days; about 3 days; about 4 days; about 5 days; about 6 days; about 7 days; about 8 days; about 9 days; about 10 days; about 11 days; about 12 days; about 13 days; about 14 days; about 21 days; about 28 days; about 30 days; about 35 days; about 40 days; about 45 days; about 50 days; about 60 days; about 120 days; about 180 days; or from any one of the recited days to any other of the recited days.

The herein disclosed amount, dose, and/or duration of the administration of zinc to a subject may be adjusted to, with phytase, cause the energy level of the subject to increase compared to the subject's energy level without phytase and zinc administration by from about 10% to 100%, for example, about 10%; about 15%; about 20%; about 25%; about 30%; about 35%; about 40%; about 45%; about 50%; about 55%; about 60%; about 65%; about 70%; about 75%; about 80%; about 85%; about 90%; about 95%; 100% or from any one of the recited percentages to any other of the recited percentages. The increase of energy may be observed following from about 1 day to about 180 days of the herein disclosed administration, for example, about 1 day; about 2 days; about 3 days; about 4 days; about 5 days; about 6 days; about 7 days; about 8 days; about 9 days; about 10 days; about 11 days; about 12 days; about 13 days; about 14 days; about 21 days; about 28 days; about 30 days; about 35 days; about 40 days; about 45 days; about 50 days; about 60 days; about 120 days; about 180 days; or from any one of the recited days to any other of the recited days.

The herein disclosed amount, dose, and/or duration of the administration of zinc to a subject may be adjusted to, with phytase, cause the pain level of the subject to decrease compared to the subject's pain level without phytase and zinc administration by from about 10% to 100%, for example, about 10%; about 15%; about 20%; about 25%; about 30%; about 35%; about 40%; about 45%; about 50%; about 55%; about 60%; about 65%; about 70%; about 75%; about 80%; about 85%; about 90%; about 95%; 100% or from any one of the recited percentages to any other of the recited percentages. The decrease in pain may be observed following from about 1 day to about 180 days of the herein disclosed administration, for example, about 1 day; about 2 days; about 3 days; about 4 days; about 5 days; about 6 days; about 7 days; about 8 days; about 9 days; about 10 days; about 11 days; about 12 days; about 13 days; about 14 days; about 21 days; about 28 days; about 30 days; about 35 days; about 40 days; about 45 days; about 50 days; about 60 days; about 120 days; about 180 days; or from any one of the recited days to any other of the recited days.

Insulin Related Disorder

Phytase may be administered to a subject with a sufficient amount of insulin prescribed by a physician. The phytase may be administered in combination with the insulin, concurrently with the insulin, or non-concurrently with the insulin. Concurrently refers to administration at the same time but not necessarily at the same part of the body. In some examples according to the present disclosure, insulin is administered from about 20 minutes prior to the administration of phytase to about 120 minutes after the administration of phytase. Alternatively, insulin is administered greater than 20 minutes prior to the administration of phytase to about greater than 120 minutes after the administration of phytase. The insulin may be combined with phytase in a composition for administration.

The amount, dosage, and/or duration of the administration of phytase to a subject may be adjusted to cause the amount of insulin administered to the subject alone, e.g., without phytase, to prevent or treat an insulin-related disorder to be reduced from about 10% to about 95%, for example, about 10%; about 15%; about 20%; about 25%; about 30%; about 35%; about 40%; about 45%; about 50%; about 55%; about 60%; about 65%; about 70%; about 75%; about 80%; about 85%; about 90%; about 95%; or from any one of the recited percentages to any other of the recited percentages. The reduction may be observed following from about 1 day to about 180 days of administration, for example, about 1 day; about 2 days; about 3 days; about 4 days; about 5 days; about 6 days; about 7 days; about 8 days; about 9 days; about 10 days; about 11 days; about 12 days; about 13 days; about 14 days; about 21 days; about 28 days; about 30 days; about 35 days; about 40 days; about 45 days; about 50 days; about 60 days; about 120 days; about 180 days; or from any one of the recited days to any other of the recited days.

Long COVID

Phytase may be administered to a subject with a sufficient amount of zinc. The phytase may be administered in combination with the zinc, concurrently with zinc, or non-concurrently with zinc. Concurrently refers to administration at the same time but not necessarily at the same part of the body. In some examples according to the present disclosure, zinc is administered from about 20 minutes prior to the administration of phytase to about 120 minutes after the administration of phytase. Alternatively, zinc is administered greater than 20 minutes prior to the administration of phytase to about greater than 120 minutes after the administration of phytase. Zinc may be combined with phytase in a composition for administration.

The amount, dose, and/or duration of the administration of phytase to a subject may be adjusted to cause the energy level of the subject to increase compared to the subject's energy level without phytase administration, with or without zinc, by from about 10% to 100%, for example, about 10%; about 15%; about 20%; about 25%; about 30%; about 35%; about 40%; about 45%; about 50%; about 55%; about 60%; about 65%; about 70%; about 75%; about 80%; about 85%; about 90%; about 95%; 100% or from any one of the recited percentages to any other of the recited percentages. The increase of energy may be observed following from about 1 day to about 180 days of administration, for example, about 1 day; about 2 days; about 3 days; about 4 days; about 5 days; about 6 days; about 7 days; about 8 days; about 9 days; about 10 days; about 11 days; about 12 days; about 13 days; about 14 days; about 21 days; about 28 days; about 30 days; about 35 days; about 40 days; about 45 days; about 50 days; about 60 days; about 120 days; about 180 days; or from any one of the recited days to any other of the recited days.

Crohn's Disease

The amount, dosage, and/or duration of the administration of phytase to a subject may be adjusted to cause the amount of Remicade™ administered to the subject alone, e.g., without phytase, to prevent or treat Crohn's disease to be reduced from about 10% to about 95%, for example, about 10%; about 15%; about 20%; about 25%; about 30%; about 35%; about 40%; about 45%; about 50%; about 55%; about 60%; about 65%; about 70%; about 75%; about 80%; about 85%; about 90%; about 95%; or from any one of the recited percentages to any other of the recited percentages. The reduction may be observed following from about 1 day to about 180 days of administration, for example, about 1 day; about 2 days; about 3 days; about 4 days; about 5 days; about 6 days; about 7 days; about 8 days; about 9 days; about 10 days; about 11 days; about 12 days; about 13 days; about 14 days; about 21 days; about 28 days; about 30 days; about 35 days; about 40 days; about 45 days; about 50 days; about 60 days; about 120 days; about 180 days; or from any one of the recited days to any other of the recited days. In some examples according to the present disclosure, the number of weeks between Remicade™ infusions was increased with the herein disclosed administration of phytase compared to when the subject was not being administered phytase, for example, by about 1 week, about 2 weeks, about 4 weeks, about 6 weeks, about 8 weeks, about 12 weeks, about 16 weeks, or more.

Ulcerative Colitis

The amount, dosage, and/or duration of the administration of phytase to a subject may be adjusted to cause the amount of Remicade™ administered to the subject alone, e.g., without phytase, to prevent or treat Ulcerative Colitis to be reduced from about 10% to about 95%, for example, about 10%; about 15%; about 20%; about 25%; about 30%; about 35%; about 40%; about 45%; about 50%; about 55%; about 60%; about 65%; about 70%; about 75%; about 80%; about 85%; about 90%; about 95%; or from any one of the recited percentages to any other of the recited percentages. The reduction may be observed following from about 1 day to about 180 days of administration, for example, about 1 day; about 2 days; about 3 days; about 4 days; about 5 days; about 6 days; about 7 days; about 8 days; about 9 days; about 10 days; about 11 days; about 12 days; about 13 days; about 14 days; about 21 days; about 28 days; about 30 days; about 35 days; about 40 days; about 45 days; about 50 days; about 60 days; about 120 days; about 180 days; or from any one of the recited days to any other of the recited days. In some examples according to the present disclosure, the number of weeks between Remicade™ infusions was increased with the herein disclosed administration of phytase compared to when the subject was not being administered phytase, for example, by about 1 week, about 2 weeks, about 4 weeks, about 6 weeks, about 8 weeks, about 12 weeks, about 16 weeks, or more.

The amount, dosage, and/or duration of the administration of phytase to a subject may be adjusted to cause the amount of steroid treatment, for example Prednisone, administered to the subject alone, e.g., without phytase, to prevent or treat Ulcerative Colitis to be reduced from about 10% to about 95%, for example, about 10%; about 15%; about 20%; about 25%; about 30%; about 35%; about 40%; about 45%; about 50%; about 55%; about 60%; about 65%; about 70%; about 75%; about 80%; about 85%; about 90%; about 95%; or from any one of the recited percentages to any other of the recited percentages. The reduction may be observed following from about 1 day to about 180 days of administration, for example, about 1 day; about 2 days; about 3 days; about 4 days; about 5 days; about 6 days; about 7 days; about 8 days; about 9 days; about 10 days; about 11 days; about 12 days; about 13 days; about 14 days; about 21 days; about 28 days; about 30 days; about 35 days; about 40 days; about 45 days; about 50 days; about 60 days; about 120 days; about 180 days; or from any one of the recited days to any other of the recited days. In some examples according to the present disclosure, the number of weeks between Remicade™ infusions was increased with the herein disclosed administration of phytase compared to when the subject was not being administered phytase, for example, by about 1 week, about 2 weeks, about 4 weeks, about 6 weeks, about 8 weeks, about 12 weeks, about 16 weeks, or more.

The amount, dosage, and/or duration of the administration of phytase to a subject may be adjusted to cause the amount of biologics treatment such as Humira™ administered to the subject alone, e.g., without phytase, to prevent or treat Ulcerative Colitis to be reduced from about 10% to about 95%, for example, about 10%; about 15%; about 20%; about 25%; about 30%; about 35%; about 40%; about 45%; about 50%; about 55%; about 60%; about 65%; about 70%; about 75%; about 80%; about 85%; about 90%; about 95%; or from any one of the recited percentages to any other of the recited percentages. The reduction may be observed following from about 1 day to about 180 days of administration, for example, about 1 day; about 2 days; about 3 days; about 4 days; about 5 days; about 6 days; about 7 days; about 8 days; about 9 days; about 10 days; about 11 days; about 12 days; about 13 days; about 14 days; about 21 days; about 28 days; about 30 days; about 35 days; about 40 days; about 45 days; about 50 days; about 60 days; about 120 days; about 180 days; or from any one of the recited days to any other of the recited days.

The amount, dosage, and/or duration of the administration of phytase to a subject may be adjusted to cause the calprotectin level of the subject to reduce compared to the calprotectin level of the subject without the administration of phytase from about 10% to about 95%, for example, about 10%; about 15%; about 20%; about 25%; about 30%; about 35%; about 40%; about 45%; about 50%; about 55%; about 60%; about 65%; about 70%; about 75%; about 80%; about 85%; about 90%; about 95%; or from any one of the recited percentages to any other of the recited percentages. The reduction may be observed following from about 1 day to about 180 days of administration, for example, about 1 day; about 2 days; about 3 days; about 4 days; about 5 days; about 6 days; about 7 days; about 8 days; about 9 days; about 10 days; about 11 days; about 12 days; about 13 days; about 14 days; about 21 days; about 28 days; about 30 days; about 35 days; about 40 days; about 45 days; about 50 days; about 60 days; about 120 days; about 180 days; or from any one of the recited days to any other of the recited days. In some examples according to the present disclosure, the amount, dosage, and/or duration of the administration of phytase to a subject is adjusted until the subject's calprotectin level is at a normal level and is maintained at the normal level, for example, from about 50 μg/mg to about 200 μg/mg.

The amount, dose, and/or duration of the administration of phytase to a subject may be adjusted to cause the pain level of the subject to decrease compared to the subject's pain level without phytase administration, with or without zinc, by from about 10% to 100%, for example, about 10%; about 15%; about 20%; about 25%; about 30%; about 35%; about 40%; about 45%; about 50%; about 55%; about 60%; about 65%; about 70%; about 75%; about 80%; about 85%; about 90%; about 95%; 100%; or from any one of the recited percentages to any other of the recited percentages. The decrease in pain may be observed following from about 1 day to about 180 days of administration, for example, about 1 day; about 2 days; about 3 days; about 4 days; about 5 days; about 6 days; about 7 days; about 8 days; about 9 days; about 10 days; about 11 days; about 12 days; about 13 days; about 14 days; about 21 days; about 28 days; about 30 days; about 35 days; about 40 days; about 45 days; about 50 days; about 60 days; about 120 days; about 180 days; or from any one of the recited days to any other of the recited days.

The present inventors surprisingly found that administering the appropriate amount of phytase to a subject, optionally with the appropriate amount of zinc, prevents or treats an immune disease, an autoimmune disease, and/or a disease caused by viral infection.

### EXAMPLES

### Example 1—Case Study with Ulcerative Colitis

Bill is a 63 year old male who was diagnosed with Ulcerative Colitis in 2017. Bill was receiving Remicade™ infusions once per month. About 3-4 days before an infusion, Bill would get a “flare up” and then had to take Prednisone beginning with 8 pills per day for a week, and then 7 pills per day for a week, etc. Once Bill got down to 3-4 pills of Prednisone per day, he would start to flare up once again, even with the Remicade™ infusions. Bill had to quit his job and was unable to leave the house without earing an adult diaper. Bill started taking 4 capsules containing 800 FTU of phytase per capsule per day and has not had a single flare up since. He takes 4 capsules (800 FTU of phytase per capsule) per day and has not taken any Prednisone since taking phytase. Prior to taking phytase, Bill had tried several diet and supplements available in the market and nothing had sufficiently worked. Now Bill can eat whatever he wants, and he feels his Colitis is in remission.

### Example 2—Case Study with Ulcerative Colitis

CT is a 36 year old farmer and mother of four children. She was diagnosed with Ulcerative Colitis at the age of 30. Before taking phytase, CT felt bloated all the time, weighed 240 lbs, always felt tired, and felt that she had little to no energy. CT's joints ached and she felt she spent much of her day on the toilet. Ct was receiving Humira™ infusions every four weeks but her disease was still poorly managed. After one week of receiving phytase, CT started to feel noticeably better, and after two months she had lost 11 pounds, her pain had gone from 8/10 to a 2/10, she had more energy, never felt bloated, and was not flaring. At CT's most recent visit with her GI doctor, her calprotectin levels had gone from 3000 down to 1500. CT has another visit in a month and has increased her dose of phytase to see if she can get her calprotectin levels closer to normal, which would be about 200. CT takes a total of 4000 FTU per day. Energy was measured on a scale from 1 to 10 with 1 being low energy and 10 being high energy. Pain was measured on a scale from 1 to 10 with 1 being low or no pain and 10 being high or severe pain.

### Example 3—Case Study with Crohn's Disease

BN is a 42 year old male who has been living with severe Crohn's disease for the past 17 years. BN has been receiving phytase for the past 3 years. Prior to receiving phytase, BN's Crohn's disease was poorly managed. CT was receiving Remicade™ infusions every four weeks, and would often flare up in the week leading up to the infusion, and for the week following the infusion. At one point, things got so bad that CT had to travel to the Mayo clinic to receive emergency help and had been admitted to the hospital several times. Since starting to receive phytase, without changing anything else, BN has been able to gain 12 pounds of lean muscle, something he had been unable to do for years. BN's doctor has gradually moved him to only receiving Remicade™ infusions every 12-14 weeks, and BN has asked if he can stop receiving the infusions altogether. BN has not had any flare ups other than when he had to travel for work and forgot to bring phytase with him, but as soon as he got home and started taking phytase again, the flare ups stopped. BN takes four capsules per day, 800 FTU per capsule for a total dose of 3200 FTU per day. BN usually takes two capsules in the morning and two capsules at lunch.

### Example 4—Case Study with Crohn's Disease

An individual with Crohn's disease, had been suffering from this disease for over 15 years. Began taking phytase, one capsule per day, (400 FTU) about 1.5 years ago. Since then, all Crohn's flare ups and attacks have completely stopped, and the doctors have moved his Remicade™ infusions to every 8 weeks instead of every 6 weeks. Individual has also been able to gain 12 pounds and has way more energy, where nothing he had tried before was able to help him gain weight. With his doctors, his goal is now to try to go 10 weeks without an infusion and, then 12 and then ideally he would like to completely stop going for the immunosuppressant Remicade™ drugs. Client has increased his dose to 800 FTU 25 per day (2 capsules).

### Example 5—Case Study with Type I Diabetes

TK is a 38 year old father of three, and a business owner. He was diagnosed with Type 1 diabetes at the age of 25, shortly after moving from Germany to the US. Typically, he felt his diabetes was poorly managed and he was taking 30 units of insulin per day. As soon as he started taking phytase, TK was able to reduce his insulin use by roughly 80% and his blood sugar stayed under control. TK takes 2-4 capsules per day, 800 PTU per capsule, for a total dose of phytase of 1600-3200 FTU per day.

### Example 6—Case Study with Type I Diabetes

BA is a 12 year old girl who was diagnosed with Type 1 diabetes at the age of 5. She has tried a number of diets and lifestyle changes, including veganism and a paleo/atkins type of diet; however, nothing has helped her control her blood sugar. BA has a loop system and a pod so that she automatically receives insulin depending on her blood sugar. BA has been taking a total dose of 800 FTU of phytase per day, for about 3 years now. Her mother finds that if BA takes more than that, her blood sugar level goes low quite quickly.

### Example 7—Case Study with Type I Diabetes

OT is a four year old girl who was diagnosed with T1DM two years ago. She takes insulin injections with each meal. OT's was having a lot of spikes in her blood sugar level and could not get it under control. While a dose of 190 FTU of phytase per meal did not sufficiently affect OT, using about 400 FTU of phytase per meal, three meals per day, OT's blood sugar stopped spiking and she was able to get it under control. Her mother found she could give OT about half the dose of insulin when she received phytase with meals. OT was even able to receive phytase instead of insulin at some meals by sprinkling phytase on her food. OT has been receiving phytase for about 4 months with consistent results.

### Example 8—Case Study with Type I Diabetes

An individual with Type 1 diabetes was able to reduce his insulin intake for carbs by 90% while taking 4 phytase capsules per day at breakfast (1600 FTU). He has increased energy and feels his body is much more sensitive to the insulin he does give it. He has been taking it for four months now with similar results.

### Example 9—Case Study with LONG COVID

MH is a 41 year old female. Since contracting COVID about a year ago, MH has had extreme fatigue, which she said was “unbearable at times” and she was unable to fulfill work duties at times, general housework was out of the question or take care of her kids. MH had shortness of breath, frequent headaches, and an elevated heart rate any time she did any type of exercise, including walking or climbing stairs. Even just standing up would make MH's heart rate jump to 130. MH had consulted with Mayo Long COVID clinic, had a chest CT, blood work, etc. and nothing was helping. MH was taking daily afternoon naps and had gone from being a marathon runner, to barely being able to walk around the block. MH started taking about 3200 FTU of phytase per day as well as about 30 mg of zinc every day, and started to feel better within a week. Over the course of a month, MH's energy went from 0/10 to 8/10. She has stopped napping and has been able to return to working full days. MH still has about one day per week where she feels fatigue, but in general, has much more energy, and her breathing and heart rate has improved.

### Example 10—Case Study with Hasimotos Disease

Patient with Hashimotos disease began treatment with phytase (1200 FTU/day) and within five weeks of starting this, was able to completely stop all of her medications, as per practitioners recommendation. Prior to this, her antibodies were extremely high and out of control and she was on high doses of Naproxene and progesterone for night sweats. She is now asymptomatic and medication free. Doctors have said that her disease is in “remission”.

### Example 11—Case Study with Stage 4 Mantle Cell Lymphoma

An individual recovering from stage 4 mantle cell lymphoma. He has undergone multiple rounds of chemotherapy and two years of C10 D1 Rituximad infusions, as well as acyclovir to keep the side effects of the infusions at bay. Because of all of the treatments, he was unable to run or ride (whereas before he was an ultra runner). He had regular cramping any time he tried to exercise, and had extreme difficulty sleeping because his legs would cramp throughout the night and affect his sleep. After one phytase capsule (400FTU), he had the best sleep he has had in months. He has continued taking phytase and no longer gets any cramps, no longer takes electrolyte supplements and has been able to return to running without any issue.

### Example 12—Case Study with Anemia and Digestive Issues

An individual with severe anemia and digestive issues. She was on a very strong constipation medication and was unable to defecate without the medication. Her ferritin was measuring at a −10 and she was having to go for weekly iron infusions. After three weeks on phytase taking two capsules per day (800FTU), her iron had rebounded to over 100, and her doctor had her stop taking the constipation medication, as she was able to defecate regularly without it.

### Example 13—Serum Negative Inflammatory Arthritis

An individual with Serum negative inflammatory arthritis. Has struggled with energy levels, concentration and digestive issues. Since starting two phytase capsules per day (800 FTU), she states that she has at least 50% more energy, sleeps better and has not had an 15 auto-immune flare up in months.

### Example 14—Effect of Phytase on Cytokine Levels in a Mouse Model of LPS-Induced Inflammation

Compliance: The discovery assays described below were conducted in accordance with the Discovery UK Standard Operating Procedures (SOP) and other methods in the testing facility at the time of the study. The study was not subjected to inspection by Quality Assurance.

Deviations: There was insufficient sample volume to analyse the cytokine composition of two Peritoneal Wash samples from the LPS+vehicle treated group (animals 2.3 and 2.7). One animal from the LPS+Phytase (low) group (animal 4.8) was terminated early due to a health issue unrelated to the study treatment. This animal was excluded from the analysis in this report.

Experimental Conditions: The study was performed in: 40 mice. Each immune-assay readout was performed in: singlicate.

Readouts: Clinical observations. Cytokine levels measured using Luminex.

Administration Schedule: Administration volume is 200 μL/mouse. All Groups are n=10. Administration schedule shown in Table 1.

TABLE 1
Administration schedule

| | | Treatments | | | |
|---|---|---|---|---|---|
| Groups | | Dose | Route | Regimen | Intervention |
| 1 | Unchallenged + | # 400 | PO | SID, Day −7 to | 0 hour, PBS, |
| | phytase (high) | FTU | | Day −1 (*7) | IP |
| 2 | LPS + vehicle | NA | PO | SID, Day −7 to | 0 hour, LPS |
| | | | | Day −1 (*7) | in saline, IP |
| 3 | LPS + phytase high | # 400 | PO | SID, Day −7 to | |
| | (Group 3) | FTU | | Day −1 (*7) | |
| 4 | LPS + phytase (low) | # 400 | PO | SID, Day −4 to | |
| | | FTU | | Day −1 (*4) | |

Aim: To investigate the effect of pre-treatment of phytase in a murine model of LPS-induced Inflammation.

Method: Adult female C57BL/6 mice were randomly allocated and allowed to acclimatise for one week. Interventions were administered according to the administrations schedule shown in Table 1. On Day 0, T=0 hour, animals were administered with 0.3 mg/kg LPS (Lipopolysaccharide from Escherichia coli 055:B5) in 0.9% saline, or 0.9% saline alone, by intra-peritoneal injection. On Day 0 at T=3 hours, animals were terminated, and peritoneal lavage was collected. Peritoneal lavage was analysed for TNFα, IL-1β, IL-6 and IL-10 content by Luminex.

Phytase Formulation: Phytase was formulated in DPBS. Phytase was weighed out into universal tubes and protected from light before the study and on the day of the administration vehicle was added and stirred to remove any large lumps, then vigorously shaken from 30-60 seconds, followed by vortexing for 30 seconds, followed. Phytase was formulated fresh every day immediately before dosing each cage and kept at RT. Time between formulation and end of dosing for each cage was less than 6 mins. The formulation was protected from light between dosing each animal.

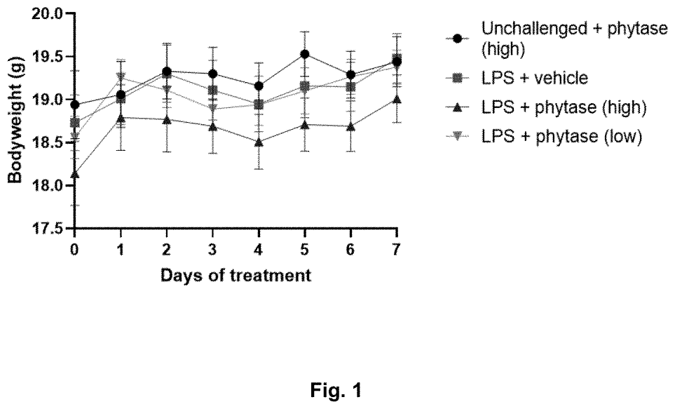

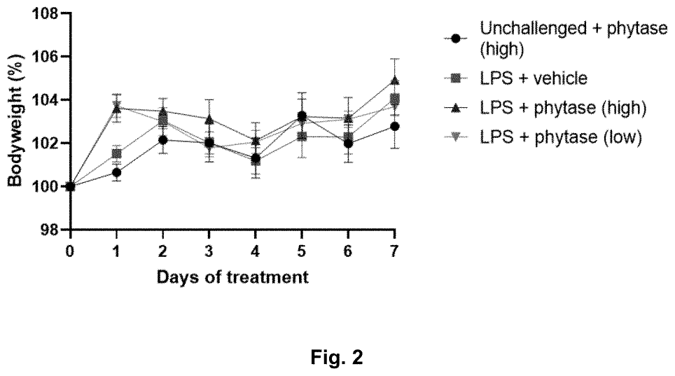

In Vivo Results—Body Weight Analysis: Animals were treated once daily with phytase (400 FTU, orally) from day 0 to day 6 or day 3-6 for LPS+phytase (low) group. Animals were weighed daily before treatment. Data is reported as mean±SEM (n=9-10 per experimental group) (FIG. 1). Animals were treated once daily with phytase (400 FTU, orally) from day 0 to day 6 or day 3-6 for LPS+phytase (low) group. Animals were weighed daily before treatment. Data is reported as mean±SEM (n=9-10 per experimental group). Data was analysed using mixed model analysis and there was no significant difference of treatment on bodyweight (FIG. 2).

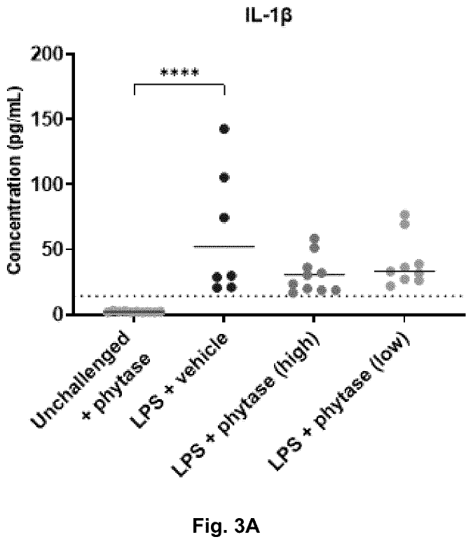

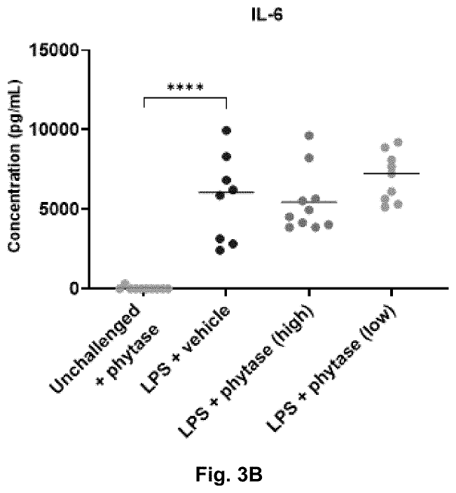

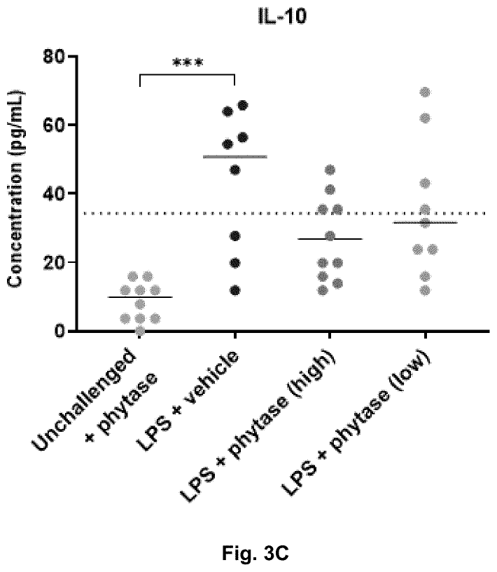

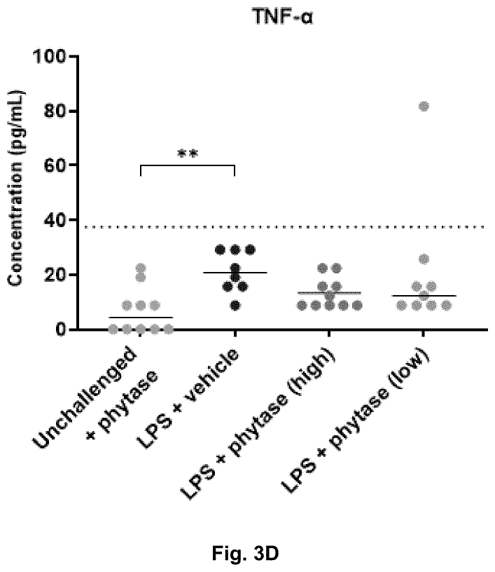

Ex Vivo Results—Cytokine Analysis: Peritoneal wash (PW) was collected from each animal following termination and cytokine analysis of IL-18 (FIG. 3A); IL-6 (FIG. 3B); IL-10 (FIG. 3C); and TNF-α (FIG. 3D) was performed by Luminex (n=8-10 per group). Samples were analysed in singlicate at a 1 in 10 dilution. Each dot represents a single animal. The group median is represented by a solid black line. The functional Lower Limit of Quantification (LLOQ) is plotted as a dotted black line. Values <LLOQ are plotted as extrapolated values where possible, 0 pg/mL where not. Statistical significance between unchallenged and LPS+Vehicle groups was determined by Mann-Whitney test (**=p<0.01, ***=p<0.001, ****=p<0.0001). Statistical significance between the LPS+Phytase and the LPS+Vehicle groups was determined using Kruskal-Wallis test followed by Dunn's multiple comparison test. No differences between these groups were found to be statistically significant.

Conclusion: Unchallenged animals treated with phytase alone did not have detectable concentrations of the cytokines tested in peritoneal wash, indicating that phytase treatment alone does not notably increase concentration of the cytokines tested in this model. Concentrations of all cytokines were detectable in the peritoneal wash (with extrapolation for IL-10 and TNF-α) following challenge with LPS alone. When compared to unchallenged animals, LPS-treatment significantly increased concentration of all cytokines compared to the Phytase+Vehicle group, markedly so for IL-18 and IL-6 and moderately so for IL-10 and TNF-α. This confirms the model performed as expected. Under the dosing regimen and conditions tested within this study, pre-treatment of animals with phytase prior to LPS-challenge did not significantly alter cytokine concentration compared to the LPS-challenged with no phytase pre-treatment group. There was a trend towards a reduction in all four cytokines measured with the highest concentration of phytase compared to LPS plus vehicle.

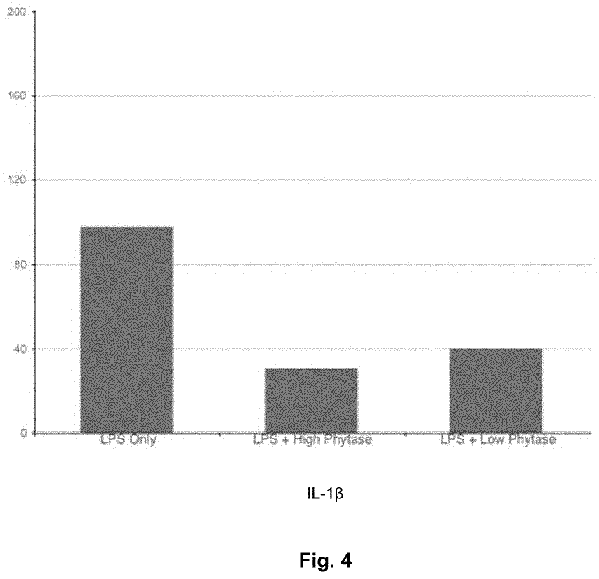

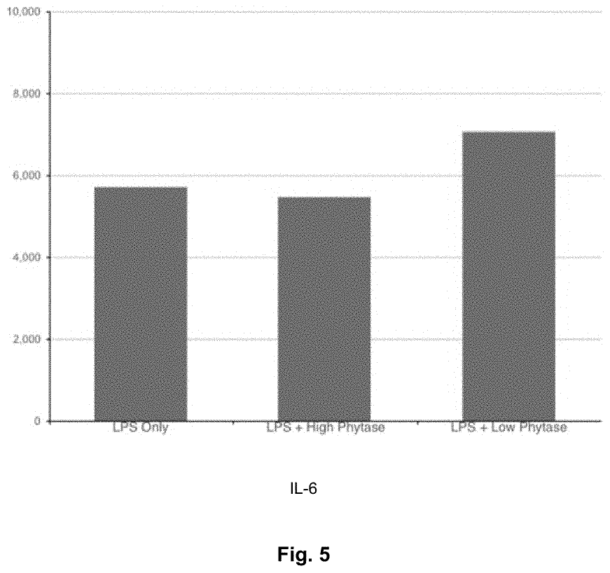

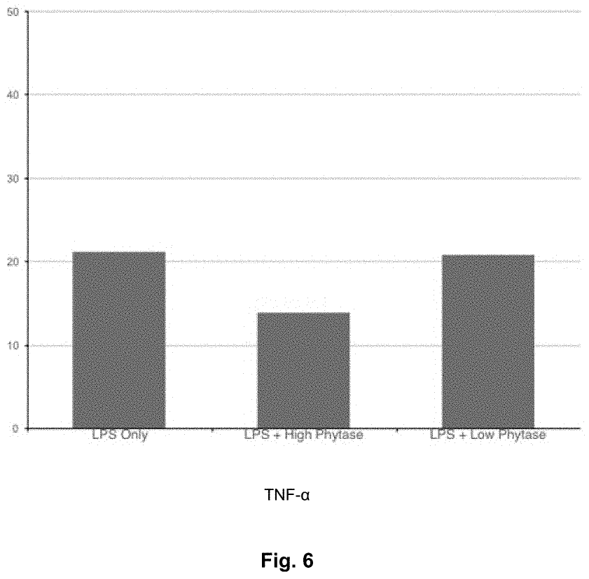

Treatment with phytase reduced levels of IL-18 by about 59% in the low dose group and about 68.7% in the high dose group (FIG. 4). Treatment with high dose phytase showed an about 4% decrease in IL-6 concentration (FIG. 5). Treatment with a high dose of phytase resulted in a statistically significant decreased (p=0.0438) of about 35% reduction in TNF-α (FIG. 6). Treatment with a low dose resulted in an about 2% reduction in TNF-α. “High dose” refers to 400 FTU of phytase administered per day per mouse for 7 days, and “low dose” refers to 400 FTU of phytase administered per day per mouse for 4 days.

In the preceding description, for purposes of explanation, numerous details are set forth in order to provide a thorough understanding of the embodiments. However, it will be apparent to one skilled in the art that these specific details are not required.

The above-described embodiments are intended to be examples only. Alterations, modifications and variations can be effected to the particular embodiments by those of skill in the art. The scope of the claims should not be limited by the particular embodiments set forth herein, but should be construed in a manner consistent with the specification as a whole.

1. A method for preventing or treating an immune disease, an autoimmune disease, and/or a disease caused by viral infection in a subject, comprising:
administering from about 400 FTU to about 5000 FTU of phytase per day to the subject,
optionally, administering from about 10 mg to about 50 mg of zinc to the subject per day.

2. The method of claim 1, wherein the phytase is administered in a dose of from about 190 FTU to about 5000 FTU, for example, about 800 FTU.

3. The method of claim 1, wherein the phytase is administered once per day, twice per day, three times per day, four times for day, five times per day, six times per day, seven times per day, eight times per day, nine times per day, 10 times per day, 11 times per day, 12 times per day, 13 times per day, 14 times per day, 15 times per day, 16 times per day, 17 times per day, 18 times per day, 19 times per day, 20 times per day, 21 times per day, 22 times per day, 23 times per day, 24 times per day, 25 times per day, 26 times per day, or more.

4. The method of claim 1, wherein zinc is administered in combination with phytase.

5. The method of claim 1, wherein zinc is administered to the subject concurrently with the phytase.

6. The method of 1, wherein zinc is administered to the subject non-concurrently with the phytase.

7. The method of claim 1, wherein the immune disease is a disease associated with elevated levels of IL-18 cytokine.

8. The method of claim 1, wherein the immune disease is a disease associated with elevated levels of IL-6 cytokine.

9. The method of claim 1, wherein the immune disease is a disease associated with elevated levels of TNF-α cytokine.

10. The method of claim 1, wherein the immune disease is Crohn's disease, Irritable Bowel Syndrome (IBS), rheumatoid arthritis, prostate cancer, systemic lupus erythematosus, Alzheimer's disease, depression, atherosclerosis, Multiple Sclerosis, ulcerative colitis, Type 1 diabetes, Type 2 diabetes, COVID-19, Long COVID, primary and secondary acquired immunodeficiency, i.e. HIV, and/or other types of inflammatory diseases.

11. The method of claim 1, wherein the autoimmune disease is Crohn's disease, Irritable Bowel Syndrome (IBS), rheumatoid arthritis, prostate cancer, systemic lupus erythematosus, Alzheimer's disease, depression, atherosclerosis, Multiple Sclerosis, ulcerative colitis, Type 1 diabetes, Type 2 diabetes, COVID-19, Long COVID, Epstein Barr, Merkel cell virus, and/or other types of inflammatory diseases.

12. The method of claim 1, wherein the disease caused by viral infection is COVID-19, Long COVID, influenza, the common cold, mumps, chickenpox, and/or herpes.

13-23. (canceled)

24. The method of claim 1, wherein the immune disease, autoimmune disease, and/or disease caused by viral infection is an insulin-related disorder.

25. The method of claim 24, wherein the insulin-related disorder is Type I diabetes or Type II diabetes.

26-27. (canceled)

28. The method of claim 24, wherein the phytase is administered in proximity to beginning of meal.

29. (canceled)

30. The method of claim 24, further comprising administering insulin to the subject.

31. (canceled)

32. The method of claim 30, wherein the insulin is administered in combination with phytase.

33. The method of claim 30, wherein the insulin is administered to the subject concurrently.

34-36. (canceled)

37. The method of claim 24, wherein the amount of insulin administered to the subject to treat the insulin-related disorder is reduced compared to treatment of the insulin-related disorder with an appropriate amount of insulin alone.

38. The method of claim 37, wherein the reduction is from about 10% to about 95%, for example, about 80%.

39. The method of claim 24, wherein the administration is oral, for example, in capsule or liquid form.

40-50. (canceled)

51. The method of claim 1, wherein the immune disease, autoimmune disease, and/or disease caused by viral infection is Long COVID.

52. The method of claim 51, further comprising administering from about 10 mg to about 50 mg of zinc to the subject per day.

53. The method of claim 52, wherein the zinc is administered to the subject in combination with the phytase.

54. The method of claim 52, wherein the zinc is administered to the subject concurrently.

55-57. (canceled)

58. The method of claim 51, wherein the phytase is administered for from about 1 day to about 360 days, or more, for example, about 30 days.

59. The method of claim 51, wherein the administration is oral, for example, in capsule or liquid form.

60. The method of claim 51, wherein the subject's energy level has increased compared to treatment of Long COVID without phytase, with or without zinc.

61-69. (canceled)

70. The method of claim 1, wherein the immune disease, autoimmune disease, and/or disease caused by viral infection is Crohn's disease.

71-72. (canceled)

73. The method of claim 70, wherein the phytase is administered in proximity to beginning of meal.

74. (canceled)

75. The method of claim 70, wherein the phytase is administered for from about 1 day to about 360 days or more, for example, about 30 days, or for about the lifetime of the subject.

76. The method of claim 70, wherein the administration is oral, for example, in capsule or liquid form.

77. The method of claim 70, wherein the amount of Crohn's antibody treatment, for example, Remicade™, administered to the subject to treat the Crohn's disease is reduced compared to treatment of Crohn's disease with an appropriate amount of Crohn's antibody treatment alone.

78. The method of claim 77, wherein the reduction is from about 25% to about 100%, for example, about 50%.

79. The method of claim 70, wherein the Crohn's disease is ileocolitis, ileitis, Gastroduodenal Crohn's Disease, Jejunoileitis, and/or Crohn's (Granulomatous) Colitis.

80-86. (canceled)

87. The method of claim 1, wherein the immune disease, autoimmune disease, and/or disease caused by viral infection is Ulcerative Colitis.

88-89. (canceled)

90. The method of claim 87, wherein the phytase is administered for from about 1 day to about 360 days or more, for example, about 30 days or for the lifetime of the subject.

91. The method of claim 87, wherein the administration is oral, for example, in capsule or liquid form.

92. The method of claim 87, wherein the amount of Ulcerative Colitis antibody treatment, for example, Remicade™, administered to the subject to treat the Ulcerative Colitis is reduced compared to treatment of Ulcerative Colitis with an appropriate amount of Ulcerative Colitis antibody treatment alone.

93. The method of claim 92, wherein the reduction is from about 10% to about 100%, for example, about 50%.

94. The method of claim 87, wherein the amount of Ulcerative Colitis steroid treatment, for example, Prednisone, administered to the subject to treat the Ulcerative Colitis is reduced compared to treatment of Ulcerative Colitis with an appropriate amount of Ulcerative Colitis steroid treatment alone.

95. The method of claim 94, wherein the reduction is from about 10% to about 100%, for example, about 50%.

96. The method of claim 87, wherein the amount of Ulcerative Colitis biologics treatment, for example, Humira™, administered to the subject to treat the Ulcerative Colitis is reduced compared to treatment of Ulcerative Colitis with an appropriate amount of Ulcerative Colitis biologics treatment alone.

97. The method of claim 96, wherein the reduction is from about 10% to about 100%, for example, about 50%.

98. The method of claim 87, wherein the subject's calprotectin level has reduced compared to treatment of Ulcerative Colitis without the phytase.

99. The method of claim 98, wherein the reduction is from about 10% to about 96%, for example, about 50%.

100. The method of claim 87, wherein the subject's pain level has decreased compared to treatment of Ulcerative Colitis without phytase.

101. The method of claim 87, wherein the Ulcerative Colitis is Ulcerative proctitis, Proctosigmoiditis, Left-sided colitis, and/or Pancolitis.

102-108. (canceled)
